(19) United States
(12) Patent Application Publication
(10) Pub. No.: US 2005/0165484 A1
(43) Pub. Date: Jul. 28, 2005

(54) Artificial disc replacement (ADR) fixation methods and apparatus

(76) Inventor: Bret A. Ferree, Cincinnati, OH (US)

Correspondence Address: John G. Posa;Gifford, Krass, Groh, Sprinkle,, Anderson & Citkowski, P.C., PO Box 7021, Troy, MI, 48007-7021, US

(21) Appl. No.: 11/041,387

(22) Filed: Jan. 24, 2005

Related U.S. Application Data
(60) Provisional application No. 60/538,179, filed on Jan. 22, 2004.

(51) Int. Cl. A61F 2/44; A61B 17/80; A61B 17/86

(52) U.S. Cl. 623/17.11; 606/71; 606/73

(57) ABSTRACT

Artificial disc replacements (ADRs), including ADRs with plate-like extensions, can be bent at the junction of the plate-like extension and the ADR Endplate (EP), which allows customization of the ADR to better fit a patient's vertebrae. A hinge joint allows customization of the ADR to fit a patient's vertebrae better. Other embodiments of the ADR contain telescoping components which allow customization of ADRs to replace two or more adjacent discs. Plate-like extensions that inter-digitate facilitate ADR insertion at two or more adjacent levels of the spine. Mechanisms to prevent screws from backing out of the plate-like projections are also disclosed. Nitinol or other shape-memory materials may be used for such purpose. Various other anti-back-out and anti-extrusion mechanisms are disclosed, all of which are applicable to non-spine applications, including long-bone plates, and total hip, knee and other joint prostheses.

### REFERENCE TO RELATED APPLICATION

This application claims priority from U.S. Provisional Patent Application Ser. No. 60/538,179, filed Jan. 22, 2004, the entire content of which is incorporated herein by reference.

### FIELD OF THE INVENTION

This invention relates generally to artificial disc replacement (ADR) and, in particular, to fixation methods and apparatus therefore.

### BACKGROUND OF THE INVENTION

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

Artificial disc replacements (ADRs) can be held in position by screws that extend into the vertebrae. Often the screws extend through plate-like projections that are placed over the anterior or lateral surface of the spine. My co-pending application Ser. No. 10/413,028 describes an ADR with a plate-like extension and screws.

ADRs are often inserted in contiguous disc spaces. Fixation of ADRs to both inferior and superior surface of the intermediate vertebra can be problematic. For example, keels inserted into the superior and inferior surface of a vertebra can weaken the vertebra. In fact, fractures of the intermediate vertebra following insertion of two or more ADRs into contiguous disc spaces have been reported.

ADR attachment mechanisms can also compromise insertion of an ADR at an adjacent level at a future surgery. For example, ADRs with plate-like portions that extend onto the anterior surfaces of the vertebrae above and below the ADR limits the surface area on the anterior surface of the vertebrae. There may not be enough surface area on the anterior surface of the vertebra to insert the plate-like portion of a second ADR.

### SUMMARY OF THE INVENTION

The present invention improves upon prior-art ADRs, including ADRs with plate-like extensions, in several respects. First, the ADR can be bent at the junction of the plate-like extension and the ADR Endplate (EP). Bending the ADR allows customization of the ADR to better fit a patient's vertebrae. The ADR may be designed to make bending the ADR easier.

ADRs according to the invention may connect the plate-like component to the ADR EP with a hinge joint. The hinge joint allows customization of the ADR to fit a patient's vertebrae better. Some embodiments of the ADR contain telescoping components which allow customization of ADRs to replace two or more adjacent discs.

The invention further teaches the use of plate-like extensions that interdigitate. The interdigitating components facilitate ADR insertion at two or more adjacent levels of the spine. The inventive ADRs may additionally incorporate a novel mechanism to prevent screws from backing out of the plate-like projections. Shape-memory components are used to hold the screws within the holes of the ADR. Nitinol or other shape-memory materials can be used. The shape-memory components could change shape as the component was placed into the body. Warm saline may be flushed over the shape-memory component to speed the shape change. Alternatively, sterile ice could be used to cause the component to change shape.

Various other anti-back-out and anti-extrusion mechanisms are disclosed, all of which are applicable to non-spine applications, including long-bone plates, and total hip, knee and other joint prostheses.

### BRIEF DESCRIPTION OF THE DRAWINGS

FIG. 1A shows an anterior view of an ADR according to the present invention and two vertebrae;

FIG. 1B is a partial sagittal view of the spine and the embodiment of the ADR drawn in FIG. 1A;

FIG. 2A is an anterior view of the spine and an alternative embodiment of the present invention;

FIG. 2B is a partial sagittal section of the embodiment of the ADR drawn in FIG. 2A;

FIG. 2C is an anterior view of two ADR EPs and the telescoping plate-like components between them;

FIG. 3 is an anterior view of an alternative embodiment of the present invention and a vertebra;

FIG. 4A is an anterior view of an alternative, telescoping configuration;

FIG. 4B is an exploded view of the embodiment of the ADR drawn in FIG. 4A;

FIG. 4C is an axial cross section through the embodiment of the ADR drawn in FIG. 4A;

FIG. 5A is an anterior view of a novel mechanism that prevents the screws from backing out of the ADR or other implanted components;

FIG. 5B is lateral view of the ring and screw drawn in FIG. 5A;

FIG. 5C is cross section of the ring and screw drawn in FIG. 5B;

FIG. 6A is an anterior view of the ring and screw drawn in FIG. 5A;

FIG. 6B is a lateral view of the ring and screw drawn in FIG. 6A;

FIG. 6C is a cross-section of the ring and screw drawn in FIG. 6A;

FIG. 7 is an anterior view of a screw and the second shape of an alternative embodiment of the shape memory ring drawn in FIG. 5A;

FIG. 8 is a cross-section of the plate-like projection from the ADR and an embodiment of the shape memory ring and screw drawn in FIG. 5A;

FIG. 9A is an anterior view of the lower half of an ADR with an alternative, shape memory, locking mechanism;

FIG. 9B is an anterior view of the embodiment of the ADR drawn in FIG. 9A;

FIG. 10A is an anterior view of the lower half of an ADR with an alternative embodiment of a shape-memory locking mechanism;

FIG. 10B is an anterior view of the embodiment of the ADR drawn in FIG. 10A;

FIG. 11A is a view of the anterior surface of the spine and a preferred embodiment of this aspect of the invention;

FIG. 11B is a sagittal cross-section of the spine and the embodiment of the present invention depicted in FIG. 11A;

FIG. 12A is an anterior view of an alternative embodiment of the invention;

FIG. 12B is a view of the superior surface of the ADR drawn in FIG. 12A;

FIG. 12C is a view of the inferior surface of the ADR drawn in FIG. 12A;

FIG. 12D is a view of the screw that is designed for insertion into the ADR drawn in FIG. 12A;

FIG. 12E is an view of the spine and the ADR drawn in FIG. 12A;

FIG. 12F is sagittal cross section of the spine and the ADR drawn in FIG. 12E;

FIG. 12G is an anterior view of the anterior surface of an alternative embodiment including small reference holes;

FIG. 12H is a view of the guide that fits onto the anterior surface of the ADR drawn in FIG. 12G;

FIG. 13A is a view of the anterior surface of the ADR drawn in FIG. 11A;

FIG. 13B is an anterior view of the ADR drawn in FIG. 13A after deforming the wall between the holes;

FIG. 13C is an anterior view of an alternative embodiment of the ADR drawn in FIG. 13A;

FIG. 13D is an anterior view of the embodiment of the ADR drawn in FIG. 13C after deforming the ADR to lock screws in the vertebrae;

FIG. 14A is an anterior view of the alternative embodiment of the invention including a rotating member;

FIG. 14B is an anterior view of the ADR drawn in FIG. 14A with the locking component rotated to prevent screw back-out;

FIG. 15A is a lateral view of an alternative embodiment including a screw cover;

FIG. 15B is a lateral view of the ADR drawn in FIG. 15A;

FIG. 16 is a lateral view of an alternative embodiment of the invention featuring keels;

FIG. 17A is a view of the vertebral surface of an alternative ADR;

FIG. 17B is a view of the vertebral surface of an alternative embodiment wherein the fixation spikes fit into slots that extend to the periphery of the ADR;

FIG. 18A is an anterior view of an alternative embodiment of the invention including screws that have a flat side;

FIG. 18B is a view of the vertebral side of the screw drawn in FIG. 18A;

FIG. 18C is a view of the side of the screw that faces the interior of the ADR when the screw is in the fully tightened position;

FIG. 18D is a view of the screw drawn in FIG. 18A and a novel screwdriver to facilitate insertion of the flat-sided screws;

FIG. 18E is a partial coronal cross section of the ADR Drawn in FIG. 18A and the threaded portion of the screwdriver;

FIG. 18F is sagittal cross section of the ADR drawn in FIG. 18E;

FIG. 18G is a lateral view of the ADR drawn in FIG. 18F with the screws drawn in the locked position;

FIG. 19A is an exploded lateral view of an alternative embodiment of the invention including an anti-extrusion component;

FIG. 19B is a sagittal cross section of the spine and the embodiment of the ADR drawn in FIG. 19A;

FIG. 19C is a view of the anterior aspect of the anti-extrusion component drawn in FIG. 19A;

FIG. 20 is a lateral view of the spine and a novel tool to determine proper ADR size;

FIG. 21 is a lateral view of the spine and a truncated conical reamer;

FIG. 22A is an anterior view of the spine and an alternative embodiment of the invention drawn in FIG. 2A; and

FIG. 22B is an anterior view of the embodiment of the invention drawn in FIG. 22A.

### DETAILED DESCRIPTION OF THE INVENTION

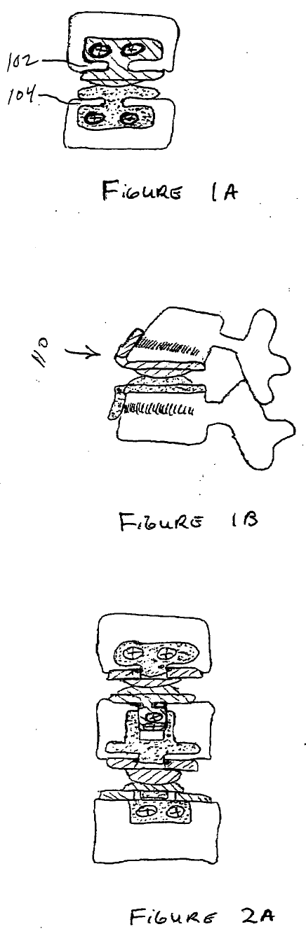

FIG. 1A is an anterior view of an ADR according to the invention and two vertebrae. The narrow areas 102, 104 connect the plate-like components to the ADR EPs facilitates bending the ADR. The screws can be locked into the ADR by C-rings incorporated in the plate-like components. The thin area of the TDR EP, between the intradiscal component and the component on the anterior surface of the vertebra, could be transected. Cutting the TDR EP allows removal of the extradiscal component during revision surgery. Removing the extradiscal component facilitates placement of a TDR at an adjacent level during a subsequent surgery. Alternatively, the strip of metal between the intradiscal and extradiscal components could be replaced with one or more cables. FIG. 1B is a partial sagittal view of the spine and the embodiment of the ADR drawn in FIG. 1A. Note that the upper ADR component 110 has bent to better fit the anterior surface of the vertebra.

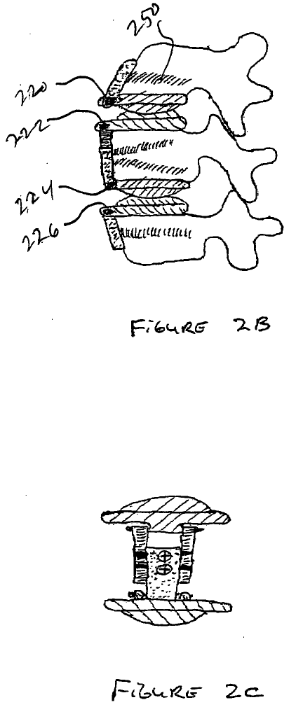

FIG. 2A is an anterior view of the spine and an alternative embodiment of the invention wherein the plate-like components of the ADRs are connected to the ADR EPs by hinge joints. The plate-like components over the central vertebra in this case also telescope to fit vertebrae of differing heights.

FIG. 2B is a partial sagittal section of the embodiment of the ADR drawn in FIG. 2A and the spine. The drawings illustrates fixation of the ADRs with different angles between the plate-like components and the ADR EPs. The various angles are allowed by motion through the hinge joints 220, 222, 224, 226. The drawing also illustrates screw fixation (i.e., 250) of the telescoping plate-like components. FIG. 2C is an anterior view of two ADR EPs and the telescoping plate-like components between them. In a preferred configuration, projections from the superior plate-like component from the inferior ADR can fit into slots in the inferior plate-like component from the superior ADR.

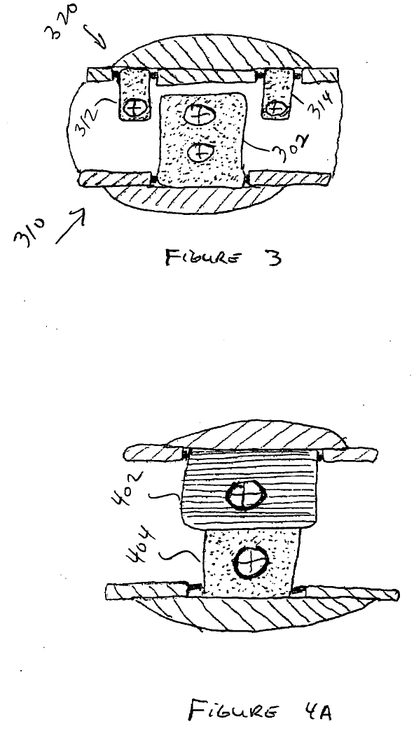

FIG. 3 is an anterior view of an alternative embodiment of the invention and a vertebra. A central plate-like component 302 of the inferior ADR 310 fits between plate-like components 312, 314 from the superior ADR 320. The plate-like components are connected to the ADR EPs by hinge joints. Alternatively, the plate-like components could project directly from the ADR EPs without the hinge joints.

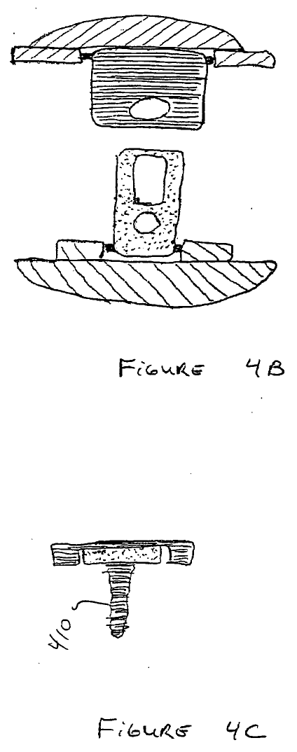

FIG. 4A is an anterior view of an alternative, telescoping configuration. The plate-like projection 402 from the superior ADR fits over the plate-like projection 404 of the inferior ADR. The extradiscal components of the TDRs could incorporate teeth. The teeth from one component could inter-digitate with the teeth of the second component. Alternatively, the extradiscal components could be fastened to one another using shape memory technology. For example, the extradiscal components could be made of nitinal. FIG. 4B is an exploded view of the embodiment of the ADR drawn in FIG. 4A. FIG. 4C is an axial cross section through the embodiment of the ADR drawn in FIG. 4A. The cross section was taken at the level of the overlapping plate-like components. A screw 410 is shown passing through both components. The screw locks the plate-like components together. The screw also connects the ADRs to the vertebra.

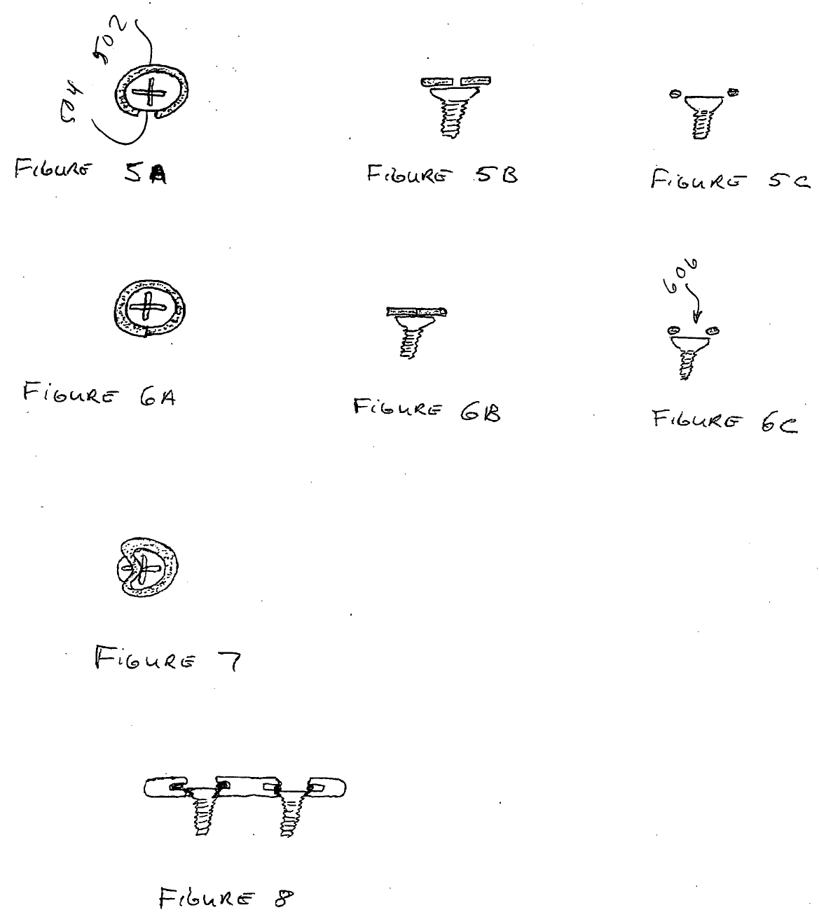

FIG. 5A is an anterior view of a novel mechanism that prevents the screws from backing out of the ADR or other implanted components. A ring 502, preferably of a shape memory material is shown over a screw 504. The ring is drawn in its first shape. The ring would be incorporated into the plate-like components of an ADR. For example, the ring could be placed into a milled out groove within a hole in the plate-like component of the ADR. FIG. 5B is lateral view of the ring and screw drawn in FIG. 5A. FIG. 5C is cross section of the ring and screw drawn in FIG. 5B. The first shape of the ring allows the screw to pass through the center of the ring.

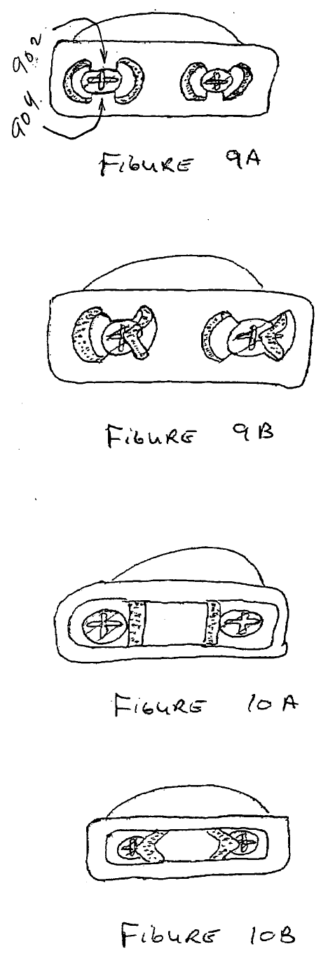

FIG. 6A is an anterior view of the ring and screw drawn in FIG. 5A. The ring has assumed its second shape. The hole formed by the ring is smaller than the diameter of the screw, in the second shape of the ring. The screw no longer passes through the hole of the ring in the second shape of the ring. FIG. 6B is a lateral view of the ring and screw drawn in FIG. 6A. FIG. 6C is a cross section of the ring and screw drawn in FIG. 6A. The narrow passage 606 in the ring in the second shape, prevents the screw from moving through the ring.

FIG. 7 is an anterior view of a screw and the second shape of an alternative embodiment of the shape memory ring drawn in FIG. 5A. A portion of the ring covers a portion of the screw in the second shape.

FIG. 8 is a cross section of the plate-like projection from the ADR and an embodiment of the shape memory ring and screw drawn in FIG. 5A. The rings fit in a groove milled into the walls of the holes within the plate-like extension. The ring on the left of the drawn is drawn in its first shape. The ring on the right of the drawing is drawn in its second shape.

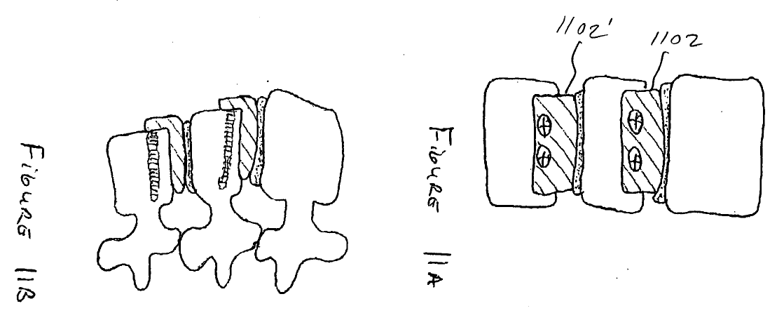

FIG. 9A is an anterior view of the lower half of an ADR with an alternative, shape memory, locking mechanism. Projections 902, 904 from the top and bottom of the holes hold the shape memory rings in the plate-like component. The rings are drawn in their first shape. FIG. 9B is an anterior view of the embodiment of the ADR drawn in FIG. 9A. The rings are drawn in their second shape.

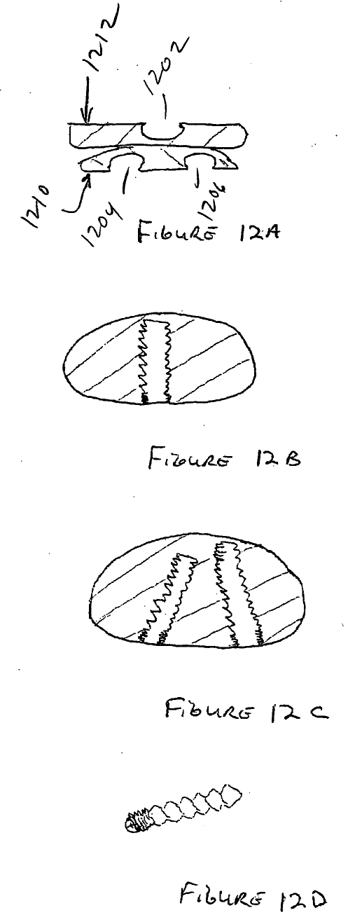

FIG. 10A is an anterior view of the lower half of an ADR with an alternative embodiment of a shape-memory locking mechanism. Shape memory wires are drawn in their first shape. FIG. 10B is an anterior view of the embodiment of the ADR drawn in FIG. 10A. The wires are drawn their second shape.

The inventions described below reside in modular attachment mechanisms that enable surgeons to check the position of the ADR before attaching the ADR to the vertebrae. Thus, a surgeon could reposition misaligned or misplaced ADRs. Current ADRs with keels attached to the ADR cannot be repositioned for fear of fracturing the vertebrae by making additional slots in the vertebrae.

FIG. 11A is a view of the anterior surface of the spine and a preferred embodiment of this aspect of the invention, wherein the plate-like portion 1102, 1102′ of the ADR is limited to the inferior ADR component. The inferior ADR component is attached to the vertebra below the ADR with screws. Mechanisms are preferably used to prevent the screws from backing out of the vertebrae. The fit between the superior and inferior ADR components prevents the superior ADR component from migrating from the disc space. The screws can converge or diverge to improve the pull strength of the ADR. The intermediate vertebra is not weakened by penetration of the superior and inferior portions of the vertebra. Placement of subsequent ADRs at adjacent level is not compromised by the attachment mechanisms of the previously inserted ADRs. FIG. 11B is a sagittal cross section of the spine and the embodiment of the invention depicted in FIG. 11A.

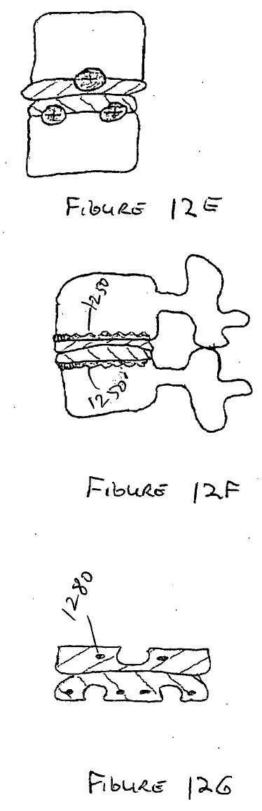

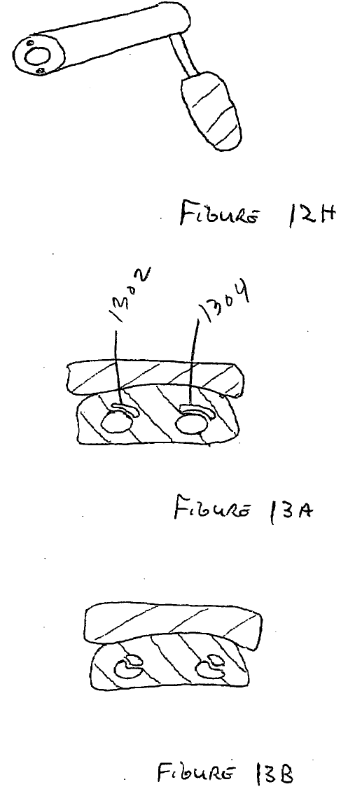

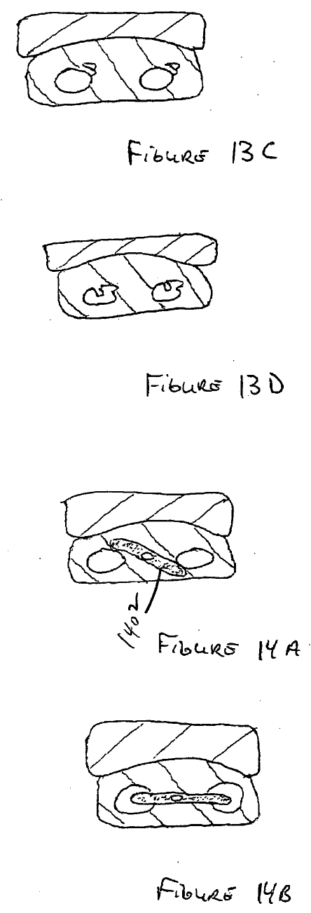

FIG. 12A is an anterior view of an alternative embodiment of the invention. The ADR has threaded holes 1202, 1204, 1206 on its vertebral-contacting surfaces 1210, 1212. The holes are open along the vertebral surfaces of the ADR. FIG. 12B is a view of the superior surface of the ADR drawn in FIG. 12A. A single thread is chased along the entire length of the hole. One or more additional threads are chased along the anterior most portion of the hole. FIG. 12C is a view of the inferior surface of the ADR drawn in FIG. 12A. The holes converge. Alternatively, the holes may be parallel or diverge. FIG. 12D is a view of the screw that is designed for insertion into the ADR drawn in FIG. 12A. The proximal portion of the screw is chased with two or more threads.

FIG. 12E is an view of the spine and the ADR drawn in FIG. 12A. The screws extend from the ADR into the vertebrae above and/or below the ADR. The screws lock to the plate. For example, the threads of the screw could vary slightly from the treads in the plate. The treads of the screw could deform slightly as the screw is tightened in the ADR, thus reversibly locking the screw to the ADR.

FIG. 12F is sagittal cross section of the spine and the ADR drawn in FIG. 12E. A portion of the screws (1250, 1250′) extend into the vertebrae.

FIG. 12G is an anterior view of the anterior surface of an alternative embodiment including small reference holes such as 1280 located around the screw holes. The reference holes are used to align a guide onto the anterior surface of the ADR. The guide can be used to drill and tap a hole in the vertebrae.

FIG. 12H is a view of the guide that fits onto the anterior surface of the ADR drawn in FIG. 12G. Projections from the guide fit into the alignment holes on the ADR. Different guides could be used to drill and tap the vertebrae. The guide used to tap the vertebrae may have internal treads to help guide the tap into the ADR and the vertebrae. A guide could also be used to insert the screws. Alternative mechanisms could be used to reversibly attach the guide to the ADR.

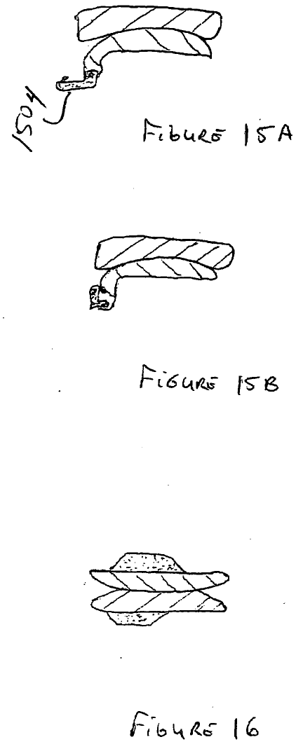

FIG. 13A is a view of the anterior surface of the ADR drawn in FIG. 11A. The drawing illustrates one possible mechanism to prevent screws from baking out of the vertebrae. Holes 1302, 1304 are seen above the screw holes. The holes accept a tool that deforms the wall between the holes and the screw holes. The walls between the holes could be moved by rotating the tool.

FIG. 13B is an anterior view of the ADR drawn in FIG. 13A after deforming the wall between the holes. The wall would be deformed after screw placement. The wall prevents the screws from backing out of the vertebrae. A second tool could be used to reposition the wall to allow screw removal. FIG. 13C is an anterior view of an alternative embodiment of the ADR drawn in FIG. 13A. FIG. 13D is an anterior view of the embodiment of the ADR drawn in FIG. 13C after deforming the ADR to lock screws in the vertebrae.

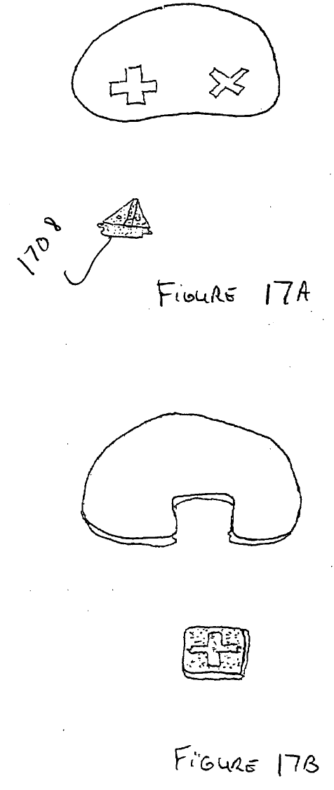

FIG. 14A is an anterior view of the alternative embodiment of the invention including a rotating member 1402 on the anterior surface of the ADR. The rotating member can be pre-attached to the ADR. Alternatively, the rotating member can be placed on the ADR after insertion of the ADR into the disc space. FIG. 14B is an anterior view of the ADR drawn in FIG. 14A with the locking component rotated to prevent screw back-out. The locking component is rotated into position after inserting the screws. The rotating member may have spring properties that force the arms of the member into the screw holes. The spring properties help prevent the locking member from spontaneously rotating into an “unlocked” position. It will be appreciated by those having mechanical skill that alternative mechanisms may be used to hold the rotating member in a “locked” position.

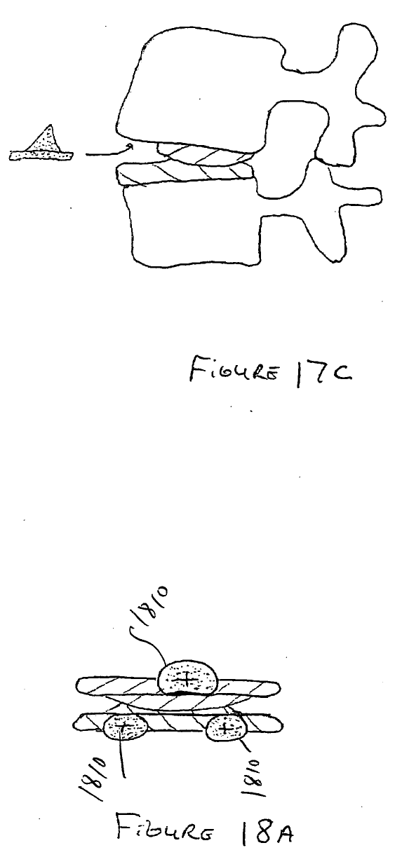

FIG. 15A is a lateral view of an alternative embodiment including a screw cover 1504 attached to the ADR with a hinge joint. The cover is drawn in its “open” position. FIG. 15B is a lateral view of the ADR drawn in FIG. 15A. The screw cover is drawn in its “closed” position. The cover prevents screws from backing out of the ADR. The cover may have a clip or other reversible mechanism to hold the cover in it “closed” position.

FIG. 16 is a lateral view of an alternative embodiment of the invention featuring keels 1602, 1604 which are limited to the anterior portion of the ADR. The smaller keels can be placed into smaller slots in the vertebrae. The strength of the vertebrae is preserved by cutting smaller slots into the vertebrae.

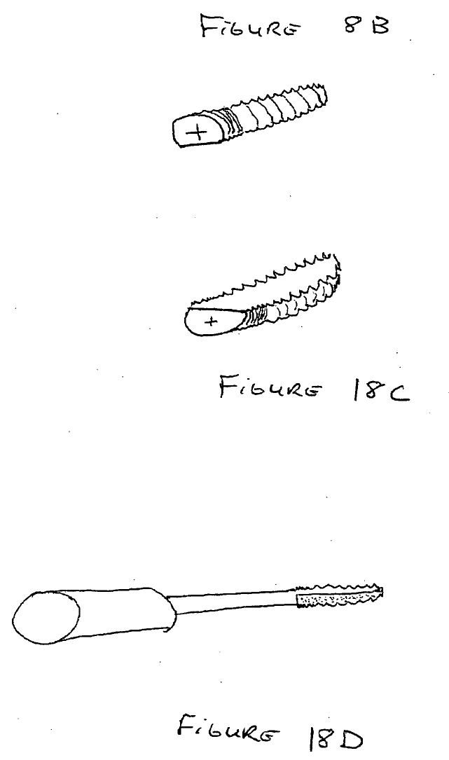

FIG. 17A is a view of the vertebral surface of an alternative ADR. Fixation spikes 1708 are inserted into the ADR after the ADR is positioned in the disc space. The spikes could attach to the ADR through the use of shape memory fastening technology.

FIG. 17B is a view of the vertebral surface of an alternative embodiment wherein the fixation spikes fit into slots that extend to the periphery of the ADR. The larger opening in the ADR facilitates spike insertion. FIG. 17C is a sagittal view of the spine and the embodiment of the ADR drawn in FIG. 17B. The fixation spike is attached to the ADR after the ADR is placed into the disc space.

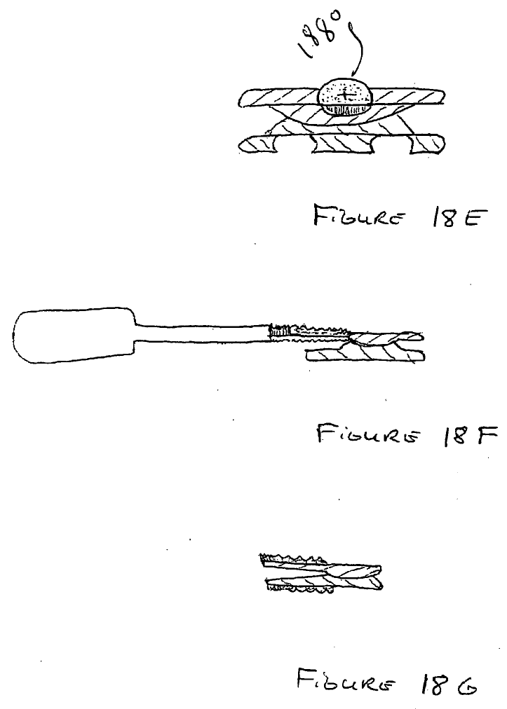

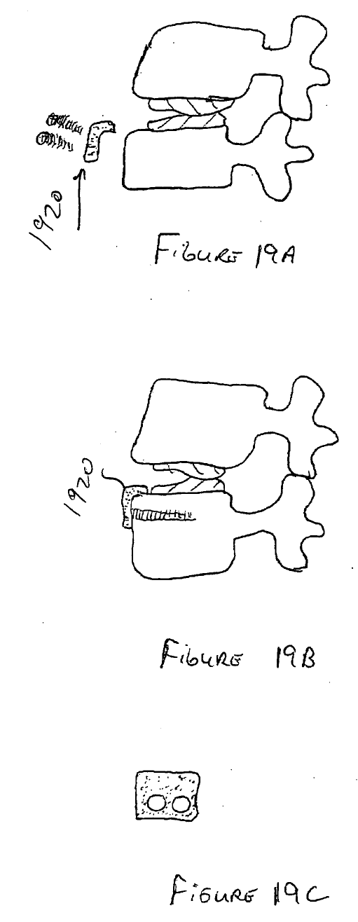

FIG. 18A is an anterior view of an alternative embodiment of the invention including screws 1810 that have a flat side. The flat side of the screws faces the interior of the ADR when the screws are fully tightened. The flat side of the screws enables the use of screws with a larger diameter without interfering with the movement of the ADR. FIG. 18B is a view of the vertebral side of the screw drawn in FIG. 18A.

FIG. 18C is a view of the side of the screw that faces the interior of the ADR when the screw is in the fully tightened position. The proximal portion of the screw preferably includes threads that deform slightly when the screw is fully tightened. Alternatively, the threads of the distal portion of the screw could deform when the screw is fully tightened. In the second embodiment, he threads in the deepest part of the hole in the ADR would be slightly different than the threads of the screw.

FIG. 18D is a view of the screw drawn in FIG. 18A and a novel screwdriver to facilitate insertion of the flat-sided screws. The tip of the screw driver fits along the flat side of the screw. The tip of the screw driver is threaded to match the threads of the flat sided screw.

FIG. 18E is a partial coronal cross section of the ADR Drawn in FIG. 18A and the threaded portion of the screwdriver. The flat sided screw is 1880. The threaded portion of the screwdriver is represented by the area of the drawing with vertical lines. The threads of the screwdriver project into the space between the ADR components when the threads of the flat sided screw are fully rotated toward the vertebra. The screwdriver can be pulled from the flat sided screw and the ADR when the screw is in the position drawn in FIG. 18E. FIG. 18F is sagittal cross section of the ADR drawn in FIG. 18E. The threads of the flat-sided screw are facing the vertebral side of the ADR. The threads of the screwdriver are drawn projecting into the interior of the ADR.

FIG. 18G is a lateral view of the ADR drawn in FIG. 18F with the screws drawn in the locked position. The screw can project the full length of the ADR. Alternatively, the screws can be limited to a portion of the ADR. Limiting the screws to the anterior surface of the ADR eliminates the need to form holes in the articulating surfaces of the ADR.

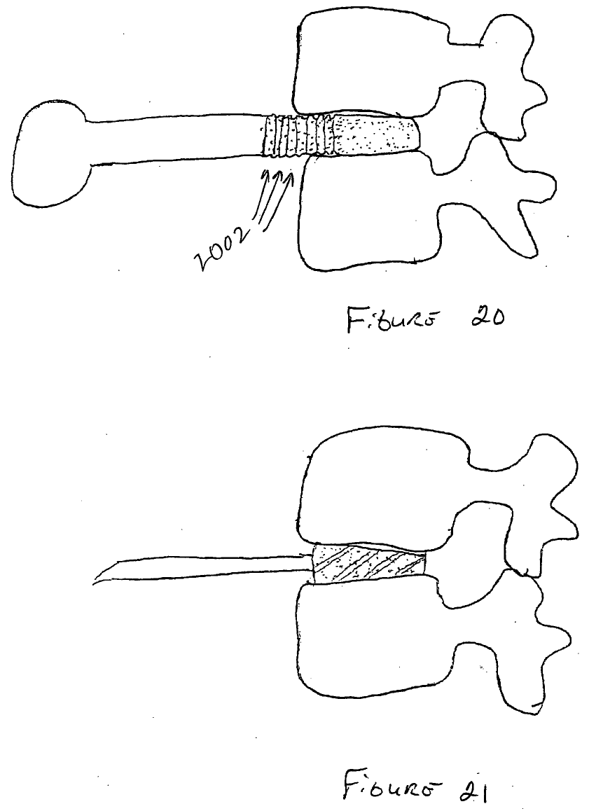

FIG. 19A is an exploded lateral view of an alternative embodiment of the invention including an anti-extrusion component 1920 drawn anterior to the spine. The component is placed onto the vertebra above or below the ADR after the ADR is placed in the disc space. The component prevents anterior extrusion of the ADR. The length of the horizontal, intradiscal, portion of the anti-extrusion is variable. A kit with anti-extrusion components with different length horizontal arms would be included with a kit of ADRs. The anti-extrusion component reduces the inventory of ADRs. ADRs with fixed plate-like components, like the ADR drawn in FIG. 11B, require a large inventory of ADRs. The sagittal placement of ADRs is critical. ADRs placed too anterior may not allow normal spinal flexion. The sagittal dimensions of vertebrae vary. Thus, kits with ADRs with fixed plate-like components must include several sizes of ADRs to assure the ADR can be placed in the proper sagittal location and the plate-like component sits against the anterior portion of the vertebra. The anti-extrusion components according to this invention lock the screws to the plate. For example, the locking mechanisms drawn in FIGS. 12-15 could be incorporated into this embodiment of the ADR.

FIG. 19B is a sagittal cross section of the spine and the embodiment of the ADR drawn in FIG. 19A. The anti-extrusion component 1920 is attached to the vertebra below the ADR. The anti-extrusion component may rest anterior the ADR, against the ADR, or attach to the anterior surface of the ADR. Attaching the anti-extrusion component to the ADR prevents migration of the ADR in all directions. FIG. 19C is a view of the anterior aspect of the anti-extrusion component drawn in FIG. 19A.

FIG. 20 is a lateral view of the spine and a novel tool to determine proper ADR size. The intradiscal portion of the tool is shaped to fit in the disc space. The tool has markings 2002 to determine the length between the end of the ADR that is closest to the spinal canal and the front of the anterior aspect of the vertebrae. The markings on the tool can be seen on radiographs of the spine. The tool helps surgeons select an ADR size that fits in the proper intradiscal location and that places the plate-like component of FIG. 11B against the anterior aspect of the vertebra. ADRs that are too short from front to back place the articulating portions of the ADR in an improper location. ADRs that are too long from front to back place the plate-like component too anterior to the vertebra.

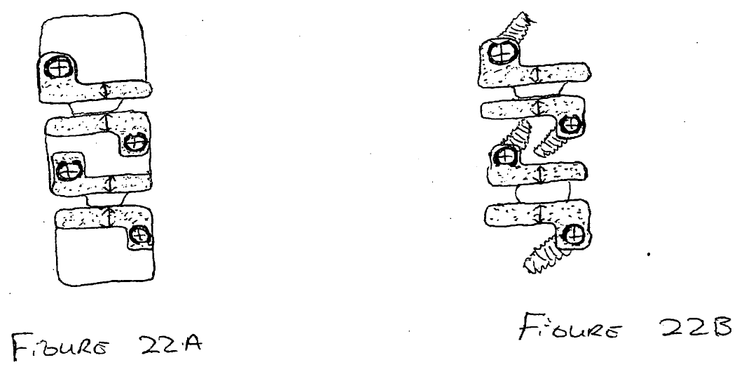

FIG. 21 is a lateral view of the spine and a truncated conical reamer that can be used to prepare the vertebral endplates. The reamer can be moved from the left to right across the disc space to improve the fit between the ADR and the vertebrae.

FIG. 22A is an anterior view of the spine and an alternative embodiment of the invention drawn in FIG. 2A. Each TDR endplate (EP) has a screw. The screw is retained in the TDR EP with anti-backout mechanism. For example, the C-ring anti-backout feature illustrated in FIG. 5A could be incorporated into the TDR EP. The anterior portion of the TDR EP have markings to help align the device. For example, the device is properly aligned when the vertical markings on the TDR EPs align with one another and align with the midline the vertebrae.

FIG. 22B is an anterior view of the embodiment of the invention drawn in FIG. 22A. The screws are oriented to avoid striking one another. For example, the screw from the inferior TDR EP could project inferiorly and possibly toward the midline. The screw from the superior TDR EP could project superiorly and possibly toward the midline. The diverging screws help prevent extrusion of the TDR.

1. In an artificial disc replacement (ADR) having a plate-like extension which overlaps at least a portion of a vertebral body, the improvement comprising:
a necking-down of the extension to facilitate bending.

2. In an artificial disc replacement (ADR) having a plate-like extension which overlaps at least a portion of a vertebral body, the improvement comprising:
a hinged portion of the extension to facilitate bending.

3. In a multi-level artificial disc replacement (ADR) situation wherein adjacent ADRs have plate-like extensions which overlap at least a portion of a respective vertebral body, the improvement comprising:
a telescoping of the plate-like extensions of the adjacent ADRs.

4. In a multi-level artificial disc replacement (ADR) situation wherein adjacent ADRs have plate-like extensions which overlap at least a portion of a respective vertebral body, the improvement comprising:
an interdigitization of the plate-like extensions of the adjacent ADRs.

5. An anti-back-out mechanism for a screw having a head used in conjunction with artificial disc replacements (ADRs) and other applications, comprising:
a deformable material having a first state that allows the screw to pass through, and a second state that prevents the screw from backing out.

6. The anti-back-out mechanism of claim 5, wherein the deformable material is a shape-memory material.

7. The anti-back-out mechanism of claim 5, wherein the deformable material is retained within an implant.

8. An anti-back-out arrangement, comprising:
an implant including a threaded channel that is open longitudinally; and.
a screw adapted to be received by the channel once the implant is in position, such that a portion of the length of the screw engages with the implant and the remaining portion of the length of the screw engages with a bone.
